(19) United States
(12) Patent Application Publication
(10) Pub. No.: US 2023/0131979 A1
(43) Pub. Date: Apr. 27, 2023

(54) DISCOMFORT GRAPH GENERATING METHOD AND SYSTEM

(71) Applicant: BEPLUS LAB. INC, Seoul (KR)

(72) Inventors: Uk LEE, Seoul (KR); Hoon Jae CHUNG, Seoul (KR); Chan YOON, Seoul (KR)

(21) Appl. No.: 17/914,723

(22) PCT Filed: Dec. 29, 2020
(86) PCT No.: PCT/KR2020/095161

§ 371 Date: Sep. 26, 2022

(30) Foreign Application Priority Data
Jun. 10, 2020 (KR) 10-2020-0070497
Jul. 27, 2020 (KR) 10-2020-0093184

(51) Int. Cl. G16H 10/20; G16H 10/60; G16H 80/00

(52) U.S. Cl. CPC G16H 10/20; G16H 10/60; G16H 80/00

(57) ABSTRACT

A method for generating a discomfort graph is disclosed. According to one aspect of the present invention, disclosed is a method for generating a discomfort graph including receiving information on a discomfort start point from a user; dividing a discomfort period from the discomfort start point to a present point into a plurality of discomfort sections; receiving from the user discomfort information obtained by quantifying discomfort levels for the respective discomfort sections; and generating a discomfort graph showing a change in the user's discomfort during the discomfort period based on the discomfort information.

### FIELD OF THE INVENTION

The present invention relates to a method for generating a discomfort graph and system thereof.

### DESCRIPTION OF THE RELATED ART

Recently, according to the development and advanced performance of computers or smart phones, computer programs or applications for providing services for user convenience in various fields have been developed, distributed, and used.

In accordance with this recent trend, even in the medical field, there is a demand for identifying the use s pain/discomfort or predicted disease and finding an appropriate department before a user visits a hospital because of the pain/discomfort or suspicion of development of diseases.

In addition, when such a user's search result is transmitted to the physician s electronic medical record (EMR), an effect that can be used as basis data for the user's treatment can be expected. Therefore, there is a growing need for methods or systems that can provide beneficial health care to people.

### DOCUMENTS OF RELATED ART

(Patent Document) KR Registered Patent No. 10-1729143 (published 2017.04.21.)

### SUMMARY OF THE INVENTION

### Technical Problem

An object of the present invention is to provide a method and system for generating a discomfort graph capable of generating and providing a discomfort graph, which can effectively identify a change pattern of discomfort during a user's discomfort period by analyzing discomfort information received from a user.

An object of the present invention is to provide a method and system for predicting developmental disease, which enables a user to effectively predict and identify his/her own developmental disease by analyzing the symptom information input from the user and generating and providing the user's predicted disease information based on the prevalence rate of the disease for the symptom.

An object of the present invention is to provide a method and system for providing basis data for diagnosis, which can provide faster and more accurate treatment by transmitting a list of multiple symptoms including the selection result of a user and the predicted disease information derived according to the user's selection of the corresponding symptom to the EMR of the attending physician of the user and using them as basis data for diagnosis.

### Technical Solution

According to one aspect of the present invention, a method for generating a discomfort graph including the steps of receiving information on a discomfort start point from a user, dividing a discomfort period from the discomfort start point to a present point into a plurality of discomfort sections, receiving from the user discomfort information obtained by quantifying discomfort levels for the respective discomfort sections, and generating a discomfort graph showing a change in the user's discomfort during the discomfort period based on the discomfort information is provided.

The plurality of discomfort sections may include a first discomfort section divided from the discomfort start point to 50% or less of the discomfort period as a period representing a reference value for the user's discomfort; a second discomfort section divided from 50% of the discomfort period to 75% or less of the discomfort period as a period representing an average value of the user's discomfort; a third discomfort section divided from 75% of the discomfort period to 90% or less of the discomfort period as a period representing a pattern of change in the user's discomfort; and a fourth discomfort section divided from 90% of the discomfort period to the present point as a period representing a current discomfort state of the user.

After the step of generating a discomfort graph, the step of analyzing the second to fourth discomfort sections of the discomfort graph to determine whether the user is in an emergency situation may be further included.

The step of analyzing the discomfort graph may be performed by calculating an average slope of the discomfort graph.

The step of analyzing the discomfort graph may include the steps of calculating a first slope with respect to a numerical value of the discomfort information of the second discomfort section to the third discomfort section; calculating a second slope with respect to a numerical value of the discomfort information of the third discomfort section to the fourth discomfort section; and calculating an absolute value of a difference between the first slope and the second slope.

After the step of analyzing the discomfort graph, the step of determining an emergency situation when the value calculated by analyzing the discomfort graph exceeds a reference value may be further included.

The step of receiving discomfort information may be performed by allowing the user to select one of the discomfort levels classified on a scale of 1 to 10.

After the step of receiving discomfort information, the step of determining an emergency situation when the discomfort information of the discomfort section including the present point is greater than or equal to a reference value may be further included.

After the step of generating a discomfort graph, the step of transmitting the discomfort graph to an electronic medical record (EMR) of the attending physician of the user may be further included.

According to another aspect of the present invention, a system for generating a discomfort graph, including a first input unit that receives information on a discomfort start point from a user; a dividing unit that divides a discomfort period from the discomfort start point to a present point into a plurality of discomfort sections; a second input unit that receives from the user discomfort information obtained by qualifying discomfort levels for the respective discomfort sections; and a generating unit that generates a discomfort graph showing a change in the user's discomfort during the discomfort period based on the discomfort information is provided.

### Advantageous Effects

According to an aspect of the present invention, a discomfort graph which can effectively identify a change pattern of discomfort during a user's discomfort period by analyzing discomfort information received from a user can be generated and provided.

According to an aspect of the present invention, it enables a user to effectively predict and identify his/her own developmental disease by analyzing the symptom information input from the user and generating and providing the user's predicted disease information based on the prevalence rate of the disease for the symptom.

According to an aspect of the present invention, it provides faster and more accurate treatment by transmitting a list of multiple symptoms including the selection result of a user and the predicted disease information derived according to the user's selection of the corresponding symptom to the EMR of the attending physician of the user and using them as basis data for diagnosis.

### BRIEF DESCRIPTION OF THE DRAWINGS

FIG. 1 is a flowchart illustrating a method for generating a discomfort graph according to a first embodiment of the present invention.

FIG. 2 is a detailed flowchart illustrating a method for generating a discomfort graph according to a first embodiment of the present invention.

FIG. 3 is a block diagram illustrating a system for generating a discomfort graph according to a second embodiment of the present invention.

FIG. 4 is a flowchart illustrating a method for predicting developmental disease according to a third embodiment of the present invention.

FIG. 5 is a detailed flowchart illustrating a method for predicting developmental disease according to a third embodiment of the present invention.

FIG. 6 is a block diagram illustrating a system for predicting developmental disease according to a fourth embodiment of the present invention.

FIG. 7 is a flowchart illustrating a method for providing basis data for diagnosis according to a fifth embodiment of the present invention.

FIG. 8 is a detailed flowchart illustrating a method for providing basis data for diagnosis according to a fifth embodiment of the present invention.

FIG. 9 is a block diagram illustrating a system for providing basis data for diagnosis according to a sixth embodiment of the present invention.

### DESCRIPTION OF REFERENCE NUMERALS

10: user

20: attending physician

30: medical expert group

100: system for generating discomfort graph

110: first input unit

120: dividing unit

130: second input unit

140: generating unit

150: analysis unit

160: determination unit

180: transmitting unit

200: system for predicting developmental disease

210: list generating unit

220: matching unit

230: score setting unit

240: symptom selecting unit

250: result deriving unit

254: first correcting unit

256: second correcting unit

258: third correcting unit

260: personal information input unit

280: transmitting unit

300: system for providing basis data for diagnosis

310: information providing unit

320: transmitting unit

330: evaluation input unit

350: correction input unit

360: update unit

### DETAILED DESCRIPTION OF THE INVENTION

Since the present invention can apply various changes and can have various embodiments, specific embodiments are illustrated in the drawings and described in detail in the detailed description. However, this is not intended to limit the present invention to specific embodiments, and it should be understood that all modifications, equivalents and substitutes included in the spirit and scope of the present invention are included. In describing the present invention, if it is determined that a detailed description of a related known technology may obscure the gist of the present invention, the detailed description thereof will be omitted.

Terms such as first, second, etc. may be used to describe various elements, but the components should not be limited by the terms. The above terms are used only for the purpose of distinguishing one component from another component.

The terms used in the present application are only used to describe specific embodiments, and are not intended to limit the present invention. The singular expression includes the plural expression unless the context clearly dictates otherwise. In the present application, it should be understood that terms such as “comprise” or “have” are intended to designate that a feature, number, step, operation, component, part, or combination thereof described in the specification exists, and this does not preclude the possibility of addition or existence of one or more other features or numbers, steps, operations, components, parts, or combinations thereof.

Hereinafter, a method for generating a discomfort graph, a method for predicting developmental disease, a method for providing basis data for diagnosis, and systems 100, 200, and 300 thereof according to the present invention will be described in detail with reference to the accompanying drawings. In doing so, the same or corresponding components are assigned the same reference numerals, and overlapping descriptions thereof will be omitted.

A method for generating a discomfort graph according to a first embodiment of the present invention will be described.

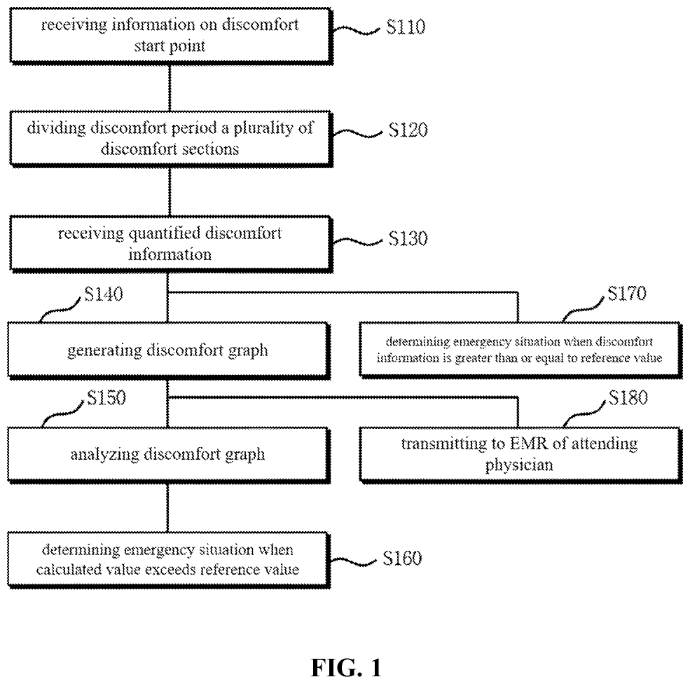

As illustrated in FIG. 1, the present embodiment provides a method for generating a discomfort graph including the steps of receiving information on a discomfort start point from a user (S110), dividing a discomfort period from the discomfort start point to a present point into a plurality of discomfort sections (S120), receiving from the user discomfort information obtained by quantifying discomfort levels for the respective discomfort sections (S130), and generating a discomfort graph showing a change in the user's discomfort during the discomfort period based on the discomfort information (S140).

According to the present embodiment as described above, it is possible to provide generating and providing the discomfort graph capable of effectively identifying the change pattern in the user's discomfort during the discomfort period by dividing the discomfort period from a corresponding point to the present point into the plurality of sections based on the information on the point at which the user's discomfort, i.e., the pain and/or discomfort is started received from the user, and receiving from the user the quantified information on a discomfort level for a corresponding section.

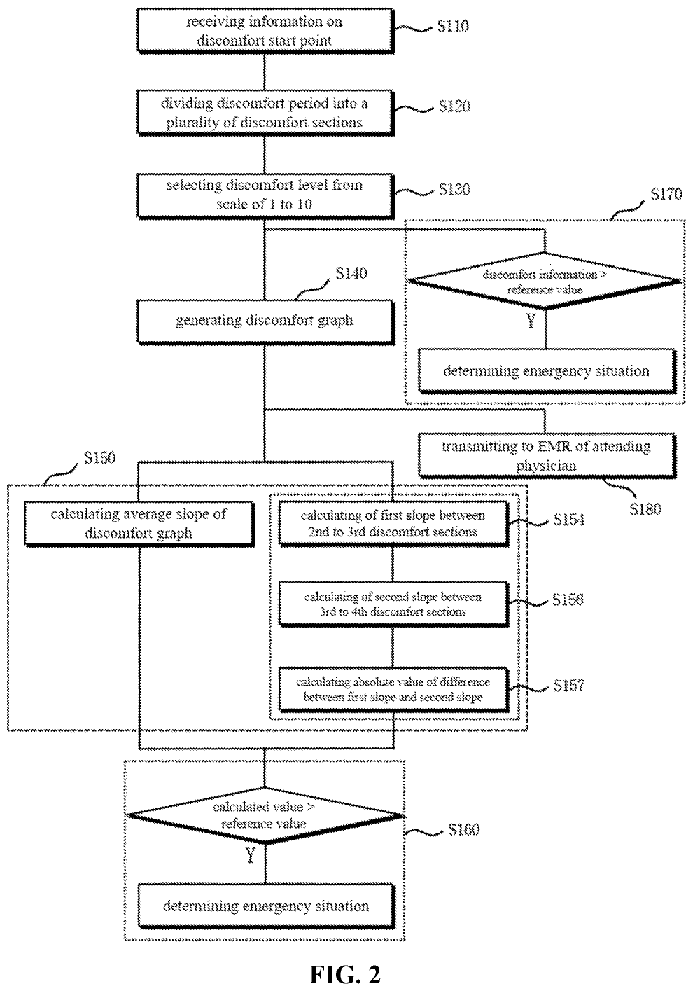

Hereinafter, each step of the method for generating a discomfort graph according to the present embodiment will be described with reference to FIGS. 1 and 2.

FIG. 1 is a flowchart illustrating a method for generating a discomfort graph according to a first embodiment of the present invention, and FIG. 2 is a detailed flowchart thereof.

In step 110, information on a discomfort start point may be input from the user.

Accordingly, the discomfort period for generating the discomfort graph, that is, a range in which the time displacement occurs on the X-axis of the discomfort graph may be set, and the basis data for generating the plurality of divided discomfort sections to be described later may be prepared.

For example, if the user started perceiving discomfort 10 days ago, the information on the discomfort start point 10 days ago is received from the user. Accordingly, the discomfort period of 10 days in total from 10 days ago to the present point is set for dividing into the plurality of discomfort sections, and a time displacement may be provided on the X-axis of the discomfort graph.

Here, the term ‘discomfort’ may be understood as meaning including physical pain and/or psychological discomfort experienced by a patient, and in the present invention, the pain having the above meaning is collectively referred as ‘discomfort’.

In step 120, the discomfort period from the discomfort start point to the present point may be divided into the plurality of discomfort sections.

In this case, as will be described later, the discomfort section may be divided according to a predetermined criterion so as to represent each meaning of the discomfort pattern that is changed during the discomfort period.

Accordingly, by receiving information on the average discomfort perceived by the user in each divided discomfort section from the user, it is possible to promote the convenience of the user's information input.

The discomfort section may be divided into a shorter length as it approaches the present point.

That is, a discomfort level is generally changed gradually after being first perceived by the user, and may become large enough for the user to wish to visit a hospital at a certain point in time. Accordingly, the discomfort section may be divided into shorter lengths as the discomfort section is closer to the present point in order to reflect such general change pattern of discomfort.

More specifically, the plurality of discomfort sections may include a first discomfort section divided from the discomfort start point to 50% or less of the discomfort period as a period representing a reference value for the user's discomfort, a second discomfort section divided from 50% of the discomfort period to 75% or less of the discomfort period as a period representing the average value of the user's discomfort, a third discomfort section divided from 75% of the discomfort period to 90% or less of the discomfort period as a period representing the patten of change in the user's discomfort, and a fourth discomfort section divided from 90% of the discomfort period to the present point as a period representing the current discomfort state of the user.

That is, the first discomfort section is a section that is temporally furthest from the present point, and the discomfort information of the corresponding section may be a reference for analyzing the discomfort information of subsequent discomfort sections.

Next, the second discomfort section is a section in which the discomfort level is gradually changed over time after the first discomfort section, and the discomfort information of the corresponding section may represent average discomfort information for the discomfort period.

In addition, the third discomfort section is a section in which a full-scale change in discomfort level occurs, and the change pattern of discomfort can be observed through discomfort information in a corresponding section.

The fourth discomfort section is a section in which the discomfort level of the user at the present point is well reflected because it is closest to the present point. Through the discomfort information of the corresponding section, it is possible to determine the user's discomfort level at the present time.

In addition, it can be understood that the division length of each section is set to reflect the general change pattern of discomfort as described above.

For example, for a discomfort period of 10 days, the first discomfort section may be divided from 10 days ago to 5 days ago, the second discomfort section may be divided from 5 days ago to 2.5 days ago, the third discomfort section may be divided from 2.5 days ago to one days ago, and the fourth discomfort section may be divided from one day ago to the present point.

In step 130, the quantified discomfort information for each discomfort section may be input from the user.

Accordingly, it is possible to generate a discomfort graph, which will be described later, by receiving a numerical input of the perceived discomfort level for each discomfort section from the user.

In other words, it is possible to prepare the magnitude displacement of the discomfort level on the X-axis of the discomfort graph through the quantified discomfort information input from the user.

More specifically, the step of receiving discomfort information (S130) may be performed by allowing the user to select one of the discomfort levels classified on a scale of 1 to 10.

That is, by providing the user with a selection range of the quantified discomfort level of 1 to 10, the user can easily input the discomfort level perceived by the user for each discomfort section.

For example, if the user feels mild discomfort in the first section, 2 points as the discomfort information may be input from the user, if the user feels slightly increased discomfort in the second section, 3 points as the discomfort information may be input from the user, if the user feels more increased discomfort in the third section, 7 points as the discomfort information may be input from the user, and if the user feels discomfort enough to feel the need to visit the hospital in the fourth section, 9 points as the discomfort information may be input from the user.

In step 140, the discomfort graph representing the change in user's discomfort during the discomfort period may be generated based on the discomfort information.

In this case, the discomfort graph may be generated in the form of a line graph in which the numerical values of discomfort information are connected by a straight line, or in the form of a curved graph reflecting the tendency of the numerical values of discomfort information.

More specifically, the discomfort graph of the above curve may be generated by analyzing the input numerical values of discomfort information based on the big data on the tendency of the discomfort information and reflecting the matched big data to the input discomfort information.

After the step of generating the discomfort graph (S140), the step of analyzing the second to fourth discomfort sections of the discomfort graph (S150) may be further included to determine whether the user is in an emergency situation.

In this case, the reason for analyzing the remaining discomfort sections other than the first discomfort section in the discomfort graph is that in the case of the first discomfort section, as described above, the user's perception of the occurrence of discomfort starts, and the full-scale change pattern of the discomfort appears from the second discomfort section. By doing so, it can be understood to effectively identify the degree of change in actual discomfort.

The step of analyzing the discomfort graph (S150) may be performed by calculating an average slope of the discomfort graph.

That is, by calculating the average slope in the corresponding sections based on the tendency indicated by the numerical values of the discomfort level in each of the second discomfort section to the fourth discomfort section, the change pattern of discomfort or its emergency can be identified.

The step of analyzing the discomfort graph (S150) may include the steps of calculating a first slope with respect to the numerical values of the discomfort information of the second discomfort section to the third discomfort section (S154), calculating a second slope with respect to the numerical values of the discomfort information of the third discomfort section to the fourth discomfort section (S156), and calculating an absolute value of a difference between the first slope and the second slope (S157).

That is, the second discomfort section to the fourth discomfort section are again divided into the second discomfort section to the third discomfort section, and the third discomfort section to the fourth discomfort section, and after calculating the slopes in the divided sections, the difference between the two slopes is calculated to obtain the absolute value of the difference, so that the discomfort graph can be analyzed.

Accordingly, it is possible to more effectively identify the pattern of changes in discomfort and its urgency from the second discomfort section indicating the average discomfort level to the third discomfort section in which the discomfort is changed and the fourth discomfort section reflecting the current discomfort level.

After the step of analyzing the discomfort graph (S150), the step of determining an emergency situation when the value calculated by analyzing the discomfort graph exceeds a reference value (S160) may be further included.

In other words, as described above, it may determine whether the user's state is currently emergency by determining whether the absolute value of the average slope or the difference in slopes for the discomfort period from the second discomfort section to the fourth discomfort section exceeds a preset reference value.

After the step of receiving the discomfort information, the step of determining an emergency situation when the discomfort information of the discomfort section including the present point is greater than or equal to a reference value (S170) may be further included.

In other words, as described above, apart from determining whether the user is in an emergency situation through the slope analysis of the discomfort graph, it may determine as an emergency when the discomfort level is greater to or equal to the reference value by intuitively comparing the discomfort level of the discomfort section including the present point with a preset reference value.

More specifically, the discomfort section including the present point may be the fourth discomfort section described above.

After the step of generating the discomfort graph (S140), the step of transmitting the discomfort graph to an electronic medical record (EMR) of the attending physician of the user (S180) may be further included.

That is, the generated discomfort graph is provided to the user so that the user can identify his or her own discomfort change, and by transmitting the discomfort graph to the EMR of the attending physician of the user, that is, the electronic medical record, the attending physician may use the discomfort graph as basis data, and this data can assist the attending physician to provide more effective treatment by identifying the changes in the user's discomfort during treatment or the current user's emergency situation.

A system for generating a discomfort graph 100 according to a second embodiment of the present invention will be described.

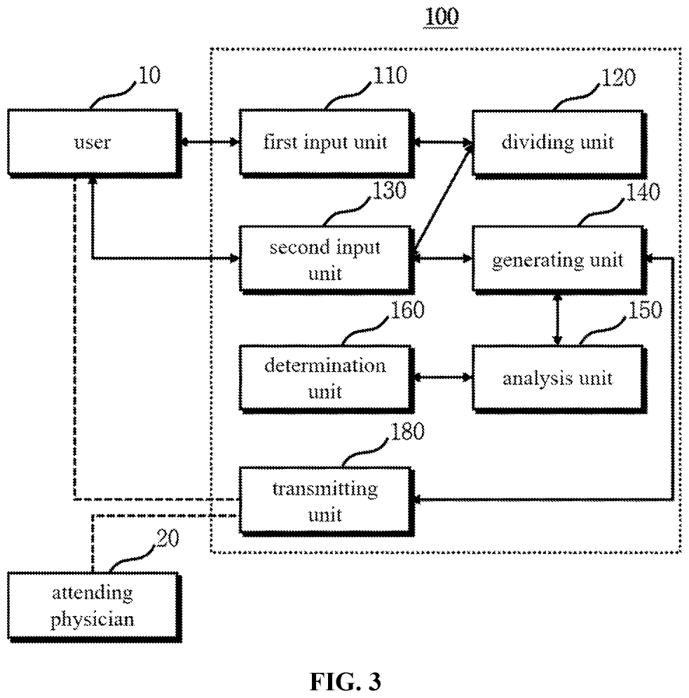

As illustrated in FIG. 3, the present embodiment provides a system for generating a discomfort graph 100 including a first input unit 110 that receives information on a discomfort start point from a user, a dividing unit 120 that divides a discomfort period from the discomfort start point to a present point into a plurality of discomfort sections, a second input unit 130 that receives from the user discomfort information obtained by qualifying discomfort levels for the respective discomfort sections, and a generating unit 140 that generates a discomfort graph showing a change in the user's discomfort during the discomfort period based on the discomfort information.

According to the present embodiment, the divisional input 120 divides the discomfort period from a corresponding point to the present point into the plurality of sections based on the information on the point at which the user's discomfort is started received from the user through the first input unit 110, and the generating unit 140 may generate the discomfort graph that can effectively identify the change pattern in the user's discomfort during the discomfort period based on the qualified information on the discomfort level for each corresponding section input through the second input unit 130, and may provide the generated discomfort graph to the user.

Hereinafter, each configuration of the system for generating a discomfort graph 100 according to the present embodiment will be described with reference to FIG. 3.

FIG. 3 is a block diagram illustrating the system for generating a discomfort graph 100 according to a second embodiment of the present invention.

The first input unit 110 may receive information on the discomfort start point from the user.

The dividing unit 120 may divide the discomfort period from the discomfort start point to the present point into a plurality of discomfort sections.

Here, the discomfort section may be divided into shorter lengths as it approaches the present point.

More specifically, the plurality of discomfort sections may include a first discomfort section divided from the discomfort start point to 50% or less of the discomfort period as a period representing a reference value for the user's discomfort, a second discomfort section divided from 50% of the discomfort period to 75% or less of the discomfort period as a period representing the average value of the user's discomfort, a third discomfort section divided from 75% of the discomfort period to 90% or less of the discomfort period as a period representing the patten of change in the user's discomfort, and a fourth discomfort section divided from 90% of the discomfort period to the present point as a period representing the current discomfort state of the user.

The second input unit 130 may receive the quantified discomfort information on the discomfort level for each discomfort section from the user.

More specifically, the second input unit 130 may allow the user to select from among discomfort levels classified on a scale of 1 to 10.

An analysis unit 150 may analyze the second to fourth discomfort sections of the discomfort graph to determine whether the user is in an emergency situation.

The analysis unit 150 may analyze the discomfort graph by calculating an average slope of the discomfort graph.

The analysis unit 150 calculates a first slope with respect to the numerical value of the discomfort information of the second discomfort section to the third discomfort section, and calculates a second slope with respect to the numerical value of the discomfort information of the third discomfort section to the fourth discomfort section. Thus, the discomfort graph can be analyzed by calculating the absolute value of the difference between the first slope and the second slope.

A determination unit 160 may determine an emergency situation when the value calculated by the analysis unit 150 analyzing the discomfort graph exceeds a reference value.

The determination unit 160 may determine an emergency situation when the discomfort information of the discomfort section including the present point is greater than or equal to a reference value.

A transmitting unit 180 may transmit the discomfort graph to the EMR of the attending physician of the user.

A method for predicting developmental disease according to a third embodiment of the present invention will be described.

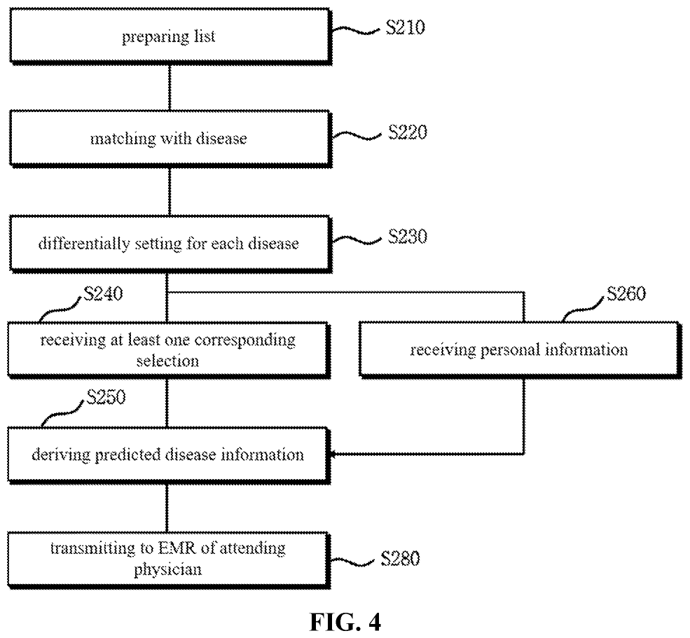

As illustrated in FIG. 4, this embodiment provides a method for predicting developmental disease including the steps of preparing a list of multiple symptoms and a list of multiple diseases, respectively (S210), matching the symptoms with the diseases related to the symptoms (S220), differentially setting correlation scores for the symptoms matched with the diseases according to prevalence rates of the diseases for the symptoms, for each disease (S230), providing a user with the list of multiple symptoms and receiving, from the user, at least one selection of the symptoms that correspond to the user (S240), and deriving predicted disease information in an order of a highest sum score by summing up scores related to the symptoms selected by the user for each disease and calculating the sum score (S250).

According to this embodiment, each symptom of the list of multiple symptoms is matched with any relevant disease of the list of multiple diseases, and in this case, the correlation score of one symptom is differentially set for each disease according to the prevalence rate between the symptom and the disease, and the predicted disease information is derived based on the sum score of the respective correlation scores for each disease according to the result of the symptom selected by the user, and the derived predicted disease information is provided to the user. Accordingly, it is possible for the user to effectively predict and identify his/her developmental disease.

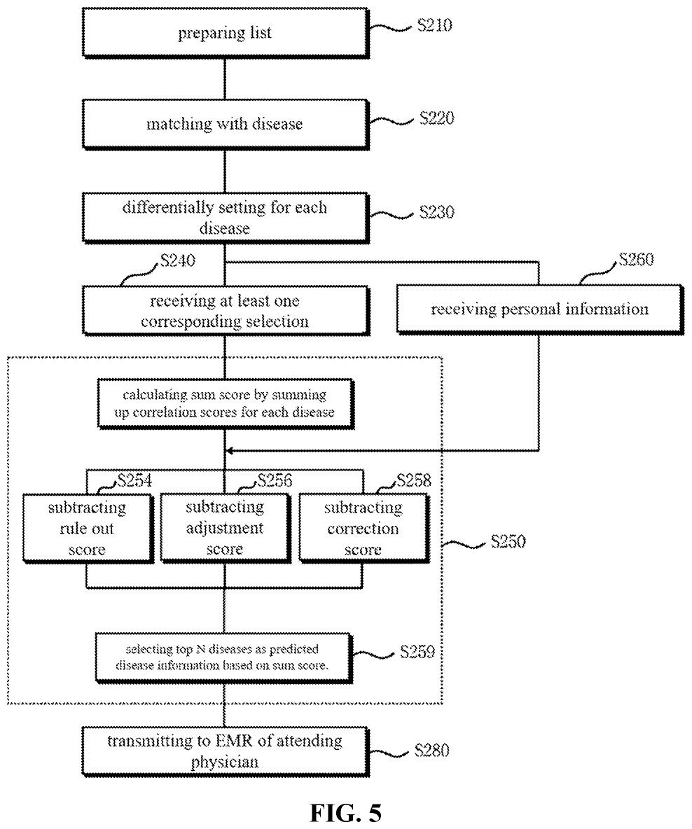

Hereinafter, each step of the method for predicting developmental disease according to the present embodiment will be described with reference to FIGS. 4 and 5.

FIG. 4 is a flowchart illustrating a method for predicting developmental disease according to a third embodiment of the present invention, and FIG. 5 is a detailed flowchart thereof.

In step 210, a list of multiple symptoms and a list of multiple diseases may be prepared, respectively.

That is, a first database may be prepared by arranging and listing possible developmental diseases, and a second database may be prepared by arranging and listing symptoms related to the diseases after excluding overlapping symptoms.

In step 220, a symptom may be matched with a disease related to the symptom.

In other words, one symptom among the multiple symptoms may be matched with the multiple diseases related to the corresponding symptom, and such matching may be repeatedly performed for all the multiple symptoms.

For example, a ‘fever’ symptom can be matched with ‘cold’, ‘migraine’, ‘typhoid’, ‘cerebral hemorrhage’, etc. related to the ‘fever’ symptom.

In step 230, a correlation score for a symptom matched with a disease may be differentially set for each disease according to a prevalence rate of the disease with respect to the symptom.

That is, the correlation score for one symptom for calculating the sum score is set differently for each matched disease, and in this case, the correlation score may be differentially set for each disease based on the prevalence rate of the disease with respect to the symptom.

Here, the prevalence rate may be understood as a percentage of people having a specific disease in a target group, and may be understood as a probability of developing a specific disease in a group of people having a specific symptom.

For example, if the prevalence rate with the ‘fever’ symptom was highest in the order of ‘cold’, ‘migraine’, ‘typhoid’ and ‘cerebral hemorrhage’, among ‘cold’, ‘migraine’, ‘typhoid’, and ‘cerebral hemorrhage’ matched with ‘fever’ symptoms, the correlation score for the ‘fever’ symptoms may be the highest with respect to the ‘cold’, and may be lowest with respect to the ‘cerebral hemorrhage’.

In step 240, the list of multiple symptoms may be provided to the user so that at least one symptom corresponding to the user may be selected by the user.

In other words, the list of multiple symptoms described above is provided to the user, and accordingly, the symptoms perceived by the user can be selected from among the symptoms of the corresponding list, which can be basis data for calculating the sum score.

In step 250, by summing up the correlation scores of the symptoms selected by the user for each disease, the sum score may be calculated, and the predicted disease information may be derived in the order of the highest sum score.

That is, for each disease including at least one symptom selected by the user, the correlation scores of the respective symptoms included in the disease are summed up, and the diseases are derived in the order of the highest score based on the sum score, so that the user can identify his/her own predicted disease.

After the step of differentially setting for each disease (S230), the step (S260) of receiving personal information including at least one of gender, age, and residential area from the user may be further included.

Accordingly, the personal information may be used as a means for correcting the sum score as will be described later, and the personal information may include at least one of gender, age, and residential area.

The step of deriving the predicted disease information (S250) may include the step of correcting the sum score by subtracting a rule out score from the sum score of the disease when the prevalence rate of the disease for the personal information is less than a first criterion (S254).

Here, the rule out means to excluding a specific disease from the result list. If the prevalence rate of a specific disease according to the user's personal information is less than the first criterion, the user can correct the sum score of the diseases by reducing the rule out score. By doing so, the accuracy of the results can be further improved by excluding the disease with a significantly low or no incidence depending on the user's personal information from the predicted disease information.

In this case, the rule out score may be set so high that the disease cannot recover the subtracted score in any case.

For example, there is a disease that occurs only in one gender according to gender. More particularly, prostate cancer, testicular cancer, cervical cancer, and ovarian cancer are diseases that occur only in one gender due to biological differences between men and women, and the prevalence rate in the other gender is significantly low or converges to 0%. If the user is male, the rule out score for cervical cancer or ovarian cancer can be subtracted from the predicted disease information and these diseases may be excluded from the predicted disease information.

The step of deriving the predicted disease information (S250) may include the step of correcting the sum score by subtracting an adjustment score from the sum score of the disease when the prevalence rate of the disease according to the personal information is less than a second criterion (S256).

That is, if the prevalence rate of a specific disease according to the user's personal information is less than the second criterion, the sum score of the corresponding disease is reduced by the adjustment score. By doing this, it is possible to prevent the disease due to the personal information with extremely low development potential, compared to the diseases due to other causes than the personal information, from being placed higher in the priority of predicted disease information, thereby improving the accuracy and efficiency of the results.

For example, the prevalence rate in the development of breast cancer in men (gender), the development of Alzheimer's disease at the age of 40 (age), or the development of malaria in Korea (region of residence) is 0% or extremely low according to individual personal information. By subtracting the adjustment score in this case, it is possible to prevent these disease from being placed higher in the priority of predicted disease information.

Here, the adjustment score may be set to increase or decrease in proportion to the prevalence rate.

The step of deriving the predicted disease information (S250) may include the step of correcting the sum score by subtracting a correction score from the sum score when the deviation between the prevalence rate of the disease due to the personal information and the prevalence rate of the disease due to causes other than the personal information is less than a third criterion (S258).

That is, if the prevalence rate of a specific disease due to the user's personal information is less than the third criterion, the sum score of the corresponding disease is corrected by subtracting the correction score. By doing this, it is possible to prevent the disease due to the personal information with extremely low development potential, compared to the diseases due to other causes than the personal information, from being placed higher in the priority of predicted disease information, thereby improving the utility and accuracy of the results.

For example, since the prevalence rate of breast cancer in men (gender) has a large deviation compared to that in women, the correction score may be subtracted in this case to prevent this disease from being placed at the top of the priority list of predicted disease information.

Here, the correction score may be set to increase or decrease in proportion to the prevalence rate.

The symptoms may include a main symptom and a sub symptom for the main symptom.

That is, the main symptom can be specifically divided into several sub symptoms, and accordingly, the accuracy of the predicted disease information can be further improved by selecting the main symptom as well as the sub symptoms by the user.

For example, the main symptom of ‘fever’ can be further subdivided into the sub symptoms such as ‘high fever’ and ‘slight fever’.

More specifically, a correlation score can be given only for the sub symptoms of the selected main symptom. In this case, first the ranking within the predicted disease information is largely determined according to whether it is related with the main symptom, and then, the correlation score is obtained according to whether it is related to the main symptom so that it is possible to determine in detail the ranking within the predicted disease information in detail.

For example, if ‘fever’ is selected as the main symptom and ‘slight fever’ is selected as the sub symptom among the sub symptoms of ‘fever’, the ‘dry eye syndrome’ is not related to ‘fever’ and cannot obtain the correlation score for the ‘slight fever’. The ‘cold’ may be accompanied by the slight fever, whereas a ‘typhoid’ is accompanied by a high fever, so ‘typhoid’ does not obtain the correlation score for the ‘slight fever’. On the other hand, the ‘cold’ acquires the correlation score for the ‘slight fever’, so ‘cold’ ' may be displayed at the top of the predicted disease information ranking.

In addition, the correlation score may be given to both the main symptom and the sub symptoms, and in this case, even for diseases that are matched to the same main symptom, the ranking of the diseases may be changed within the predicted disease information depending on additional acquisition of the correlation score according to the degree of relationship with the sub symptoms for the main symptom.

For example, if the ‘fever’ is selected as the main symptom and the ‘slight fever’ is selected as the sub symptom among the sub symptoms of the ‘fever’, the ‘cold’ has a higher correlation score for the ‘fever’ than ‘typhoid’, so the ‘cold’ can have a higher ranking in the predicted disease information. Since the ‘cold’ can be accompanied by the slight fever whereas the ‘typhoid’ is accompanied by a high fever, the difference in the sum score of the two diseases according to the correlation score for the ‘slight fever’ increases. Thus, a clear difference may occur in the ranking between the ‘cold’ and the ‘typhoid’ in the predicted disease information.

The step of deriving the predicted disease information (S250) may be performed by selecting the top N diseases as the predicted disease information based on the sum score.

That is, the user may need to be provided with only the most likely predicted disease information. In this case, the utility of the provided information can be further improved by providing the user with only N selected predicted disease information, such as the top 1, 2, 3, etc., in the order of the highest score, based on the sum score.

More specifically, before the step of deriving the predicted disease information (S250), the step (S259) of receiving a selection from the user of the number of higher-order diseases to be displayed as the predicted disease information based on the sum score may be further included.

After the step of deriving the predicted disease information (S250), the step (S280) of transmitting the list of multiple symptoms including the selection result of the user and the predicted disease information to the EMR of the attending physician of the user may be further included.

That is, the derived predicted disease information is provided to the user so that the user can identify his/her own developmental disease, and by transmitting the list of multiple symptoms including the selection result of the user and the predicted disease information to the EMR of the attending physician of the user, it is possible to assist the attending physician to provide more effective treatment by using the predicted disease information as basic data.

A system for predicting developmental disease 200 according to a fourth embodiment of the present invention will be described.

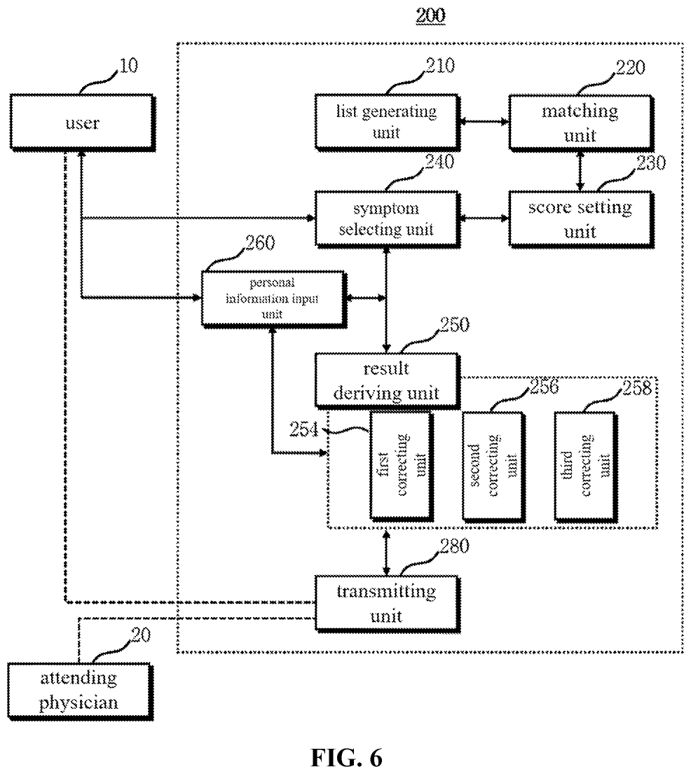

As illustrated in FIG. 6, this embodiment provides a system for predicting developmental disease including a list generating unit 210 that prepares a list of multiple symptoms and a list of multiple diseases, respectively, a matching unit 220 that matches the symptoms with the diseases related to the symptoms, a score setting unit 230 that differentially sets correlation scores for the symptoms matched with the diseases according to prevalence rates of the diseases for the symptoms, for each disease, a symptom selecting unit 240 that provides a user with the list of multiple symptoms and receives, from the user, at least one selection of the symptoms that correspond to the user, and a result deriving unit 250 that derives predicted disease information in an order of a highest sum score by summing up scores related to the symptoms selected by the user for each disease and calculating the sum score.

According to this embodiment, each symptom of the list of multiple symptoms is matched with any relevant disease of the list of multiple diseases in the matching unit 220, and in this case, the correlation score of one symptom is differentially set for each disease according to the prevalence rate between the symptom and the disease in the score setting unit 230, and the predicted disease information is derived, in the result deriving unit 250, based on the sum score of the respective correlation scores for each disease according to the result of the symptom selected by the user in the symptom selecting unit 240, and the derived predicted disease information is provided to the user. Accordingly, it is possible for the user to effectively predict and identify his/her disease.

Hereinafter, each configuration of the system for predicting developmental disease 200 according to the present embodiment will be described with reference to FIG. 6.

FIG. 6 is a block diagram illustrating the system for predicting developmental disease 200 according to a fourth embodiment of the present invention.

The list generating unit 210 may prepare a list of multiple symptoms and a list of multiple diseases, respectively.

The matching unit 220 may match a symptom with a disease related to the symptom.

The score setting unit 230 may differentially set a correlation score for a symptom matched with a disease for each disease according to a prevalence rate of the disease with respect to the symptom.

The symptom selecting unit 240 may provide the list of multiple symptoms to the user so that at least one symptom corresponding to the user may be selected by the user.

The result deriving unit 250 may derive the predicted disease information in the order of the highest sum score by summing up the correlation scores of the symptoms selected by the user for each disease and calculating the sum score.

After the step of differentially setting for each disease, a personal information input unit 260 may receive personal information including at least one of gender, age, and residential area from the user.

The result deriving unit 250 may include a first correcting unit 254 that corrects the sum score by subtracting a rule out score from the sum score of the disease when the prevalence rate of the disease for the personal information is less than a first criterion.

The result deriving unit 250 may include a second correcting unit 256 that corrects the sum score by subtracting an adjustment score from the sum score of the disease when the prevalence rate of the disease according to the personal information is less than a second criterion.

The result deriving unit 250 may include a third correcting unit 258 that corrects the sum score by subtracting a correction score from the sum score when the deviation between the prevalence rate of the disease due to the personal information and the prevalence rate of the disease due to causes other than the personal information is less than a third criterion.

The symptoms may include a main symptom and a sub symptom for the main symptom.

The result deriving unit 250 may select the top N diseases as the predicted disease information based on the sum score.

A transmitting unit 280 may transmit the list of multiple symptoms including the selection result of the user and the predicted disease information to the EMR of the attending physician of the user.

A method for providing basis data for diagnosis according to a fifth embodiment of the present invention will be described.

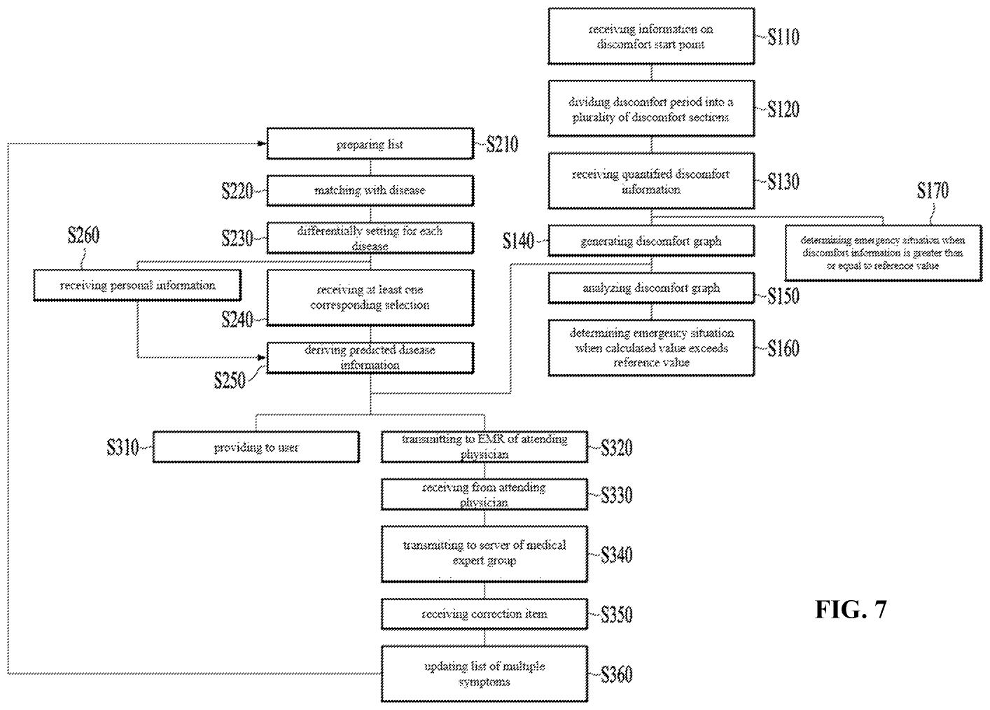

According to the present embodiment, as illustrated in FIG. 7, a method for providing basis data for diagnosis including the steps of preparing a list of multiple symptoms and a list of multiple diseases, respectively (S210), matching the symptoms with the diseases related to the symptoms (S220), differentially setting correlation scores for the symptoms matched with the diseases according to prevalence rates of the diseases for the symptoms, for each disease (S230), providing a user with the list of multiple symptoms and receiving, from the user, at least one selection of the symptoms that correspond to the user (S240), deriving predicted disease information in an order of a highest sum score by summing up scores related to the symptoms selected by the user for each disease and calculating the sum score (S250), providing the selection result of the user and the predicted disease information to the user (S310), and transmitting to an electronic medical record (EMR) of an attending physician of the user the list of the multiple symptoms including the selection result of the user and the predicted disease information (S320).

According to this embodiment, each symptom of the list of multiple symptoms is matched with any relevant disease of the list of multiple diseases, and in this case, the correlation score of one symptom is differentially set for each disease according to the prevalence rate between the symptom and the disease, and the predicted disease information is derived based on the sum score of the respective correlation scores for each disease according to the result of the symptom selected by the user, and the derived predicted disease information is provided to the user. Accordingly, it is possible for the user to effectively predict and identify his/her developmental disease. Furthermore, by transmitting the list of multiple symptom including the selection result of the user and the predicted disease information to EMR of the attending physician of the user for using as basis data for diagnosis, it is possible to provide faster and more accurate treatment.

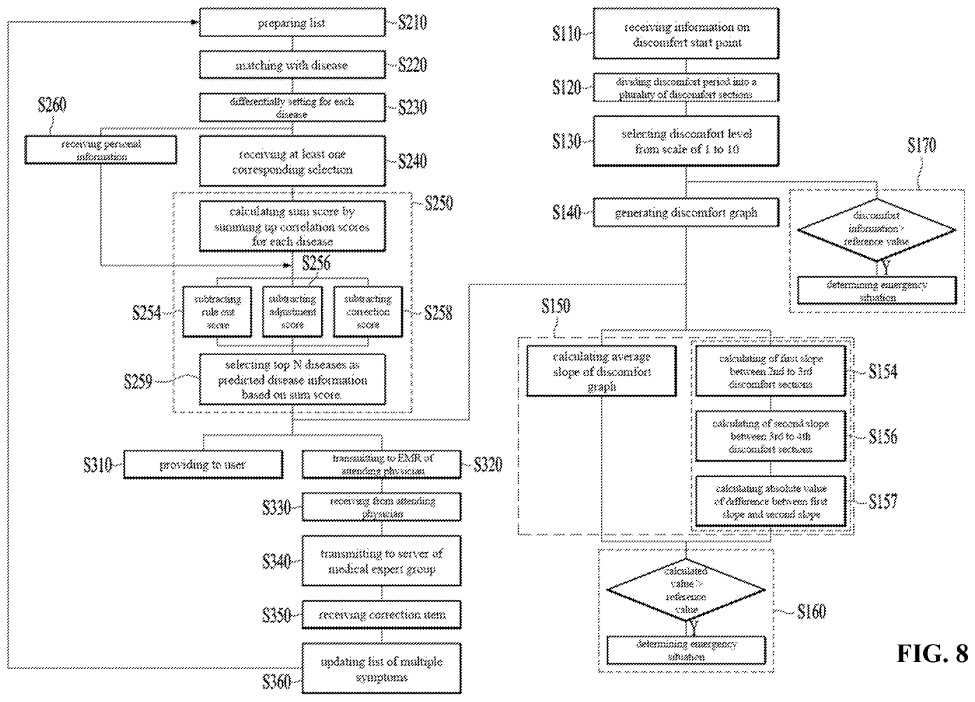

Hereinafter, each step of the method for providing basis data for diagnosis according to the present embodiment will be described with reference to FIGS. 7 and 8.

FIG. 7 is a flowchart illustrating a method for providing basis data for diagnosis according to a fifth embodiment of the present invention, and FIG. 8 is a detailed flowchart thereof.

In step 210, a list of multiple symptoms and a list of multiple diseases may be prepared, respectively.

In step 220, a symptom may be matched with a disease related to the symptom.

In step 230, a correlation score for a symptom matched with a disease may be differentially set for each disease according to a prevalence rate of the disease with respect to the symptom.

In step 240, the list of multiple symptoms may be provided to the user so that at least one symptom corresponding to the user may be selected by the user.

In step 250, by summing up the correlation scores of the symptoms selected by the user for each disease, the sum score may be calculated, and the predicted disease information may be derived in the order of the highest sum score.

After the step of differentially setting for each disease (S230), the step (S260) of receiving personal information including at least one of gender, age, and residential area from the user may be further included.

The step of deriving the predicted disease information (S250) may include the step of correcting the sum score by subtracting a rule out score from the sum score of the disease when the prevalence rate of the disease for the personal information is less than a first criterion (S254).

The step of deriving the predicted disease information (S250) may include the step of correcting the sum score by subtracting an adjustment score from the sum score of the disease when the prevalence rate of the disease according to the personal information is less than a second criterion (S256).

The step of deriving the predicted disease information (S250) may include the step of correcting the sum score by subtracting a correction score from the sum score when the deviation between the prevalence rate of the disease due to the personal information and the prevalence rate of the disease due to causes other than the personal information is less than a third criterion (S258).

The symptoms may include a main symptom and a sub symptom for the main symptom.

The step of deriving the predicted disease information (S250) may be performed by selecting the top N diseases as the predicted disease information based on the sum score.

In step 320, the list of multiple symptoms including the selection result of the user and the predicted disease information may be transmitted to the EMR of the attending physician of the user.

Accordingly, it is possible for the attending physician to use the list of multiple symptoms including the selection result of the user and the predicted disease information as basis data for diagnosis to enable faster and more accurate treatment.

Before the step of providing to the user (S310), the steps of receiving information on a discomfort start point from a user (S110), dividing a discomfort period from the discomfort start point to a present point into a plurality of discomfort sections (S120), receiving from the user discomfort information obtained by quantifying discomfort levels for the respective discomfort sections (S130), and generating a discomfort graph showing a change in the user's discomfort during the discomfort period based on the discomfort information (S140) may be further included. The step of transmitting to the EMR of the attending physician of the user (S320) may be performed to further transmit the discomfort graph to the EMR of the attending physician of the user.

Each step will be described as follows.

In step 110, information on a discomfort start point may be input from the user.

In step 120, the discomfort period from the discomfort start point to the present point may be divided into the plurality of discomfort sections.

The discomfort section may be divided into a shorter length as it approaches the present point.

The plurality of discomfort sections may include a first discomfort section divided from the discomfort start point to 50% or less of the discomfort period as a period representing a reference value for the user's discomfort; a second discomfort section divided from 50% of the discomfort period to 75% or less of the discomfort period as a period representing an average value of the user's discomfort; a third discomfort section divided from 75% of the discomfort period to 90% or less of the discomfort period as a period representing a patten of change in the user's discomfort; and a fourth discomfort section divided from 90% of the discomfort period to the present point as a period representing a current discomfort state of the user.

In step 130, quantified discomfort information for each discomfort section may be input from the user.

More particularly, the step of receiving discomfort information may be performed by allowing the user to select one of the discomfort levels classified on a scale of 1 to 10.

In step 140, the discomfort graph representing the change in user's discomfort during the discomfort period may be generated based on the discomfort information.

After the step of generating the discomfort graph (S140), the step of analyzing the second to fourth discomfort sections of the discomfort graph (S150) may be further included to determine whether the user is in an emergency situation.

The step of analyzing the discomfort graph (S150) may be performed by calculating an average slope of the discomfort graph.

The step of analyzing the discomfort graph (S150) may include the steps of calculating a first slope with respect to the numerical values of the discomfort information of the second discomfort section to the third discomfort section, calculating a second slope with respect to the numerical values of the discomfort information of the third discomfort section to the fourth discomfort section, and calculating an absolute value of a difference between the first slope and the second slope.

After the step of analyzing the discomfort graph (S150), the step of determining an emergency situation when the value calculated by analyzing the discomfort graph exceeds a reference value (S160) may be further included.

After the step of receiving the discomfort information (S130), the step of determining an emergency situation when the discomfort information of the discomfort section including the present point is greater than or equal to a reference value (S170) may be further included.

The step of transmitting to the EMR of the attending physician (S320) may be performed to further transmit the discomfort graph to the EMR of the attending physician of the user.

After transmitting to the EMR of the attending physician (S320), the step of receiving from the attending physician a result on whether or not the list of multiple symptoms meets an evaluation criteria (S330) may be further included.

That is, it is possible to determine whether or not to correct and update the list of multiple symptoms by receiving from the attending physician whether or not the list of multiple symptoms meets a preset evaluation criteria, as will be described later.

In more detail, the step (S330) of receiving from the attending physician the result may be performed based on the diagnosis result for the user by the attending physician.

In this case, when the attending physician performs diagnosis based on the list of multiple symptoms including the selection result of the user and the predicted disease information and evaluates the list of multiple symptoms and the predicted disease information based on the diagnosis result, if the list is not reasonable or if the predicted disease information and the actually diagnosed disease do not match, the result that the list of multiple symptoms does not meet the evaluation criteria may be received from the attending physician.

In addition, after the step (S330) of receiving from the attending physician the result, the step of transmitting the list of multiple symptoms to a server of a designated medical expert group if the list of multiple symptoms does not meet the evaluation criteria (S340) and the step of receiving a correction item for the list of multiple symptoms from the medical expert group (S350) may be further included.

Accordingly, the list of multiple symptoms that does not meet the evaluation criteria may be corrected by the medical expert group as collective intelligence, and accordingly, as described later, the list of multiple symptoms is updated to further improve predictivity of disease.

In more detail, the step (S330) of receiving from the attending physician the result may be performed to receive an opinion of the attending physician regarding the result below the evaluation criteria if the list of multiple symptoms does not meet the evaluation criteria. The step (S340) of transmitting to the server of the medical expert group may be performed to further transmit the opinion to the server of the medical expert group.

In this case, the opinion of the attending physician on the fact result that the list of multiple symptoms does not meet the evaluation criteria can serve as a guideline for the correction by the medical expert group, so that the correction by the medical expert group can be made more quickly and effectively.

After the step (S350) of receiving the correction item, the step of updating the list of multiple symptoms by correcting the list of multiple symptoms according to the correction item (S360) may be further included.

In this case, since the list of multiple symptom is updated, it is possible to further improve the predictivity of disease to be performed.

A system for providing basic data for diagnosis according to a sixth embodiment of the present invention will be described.

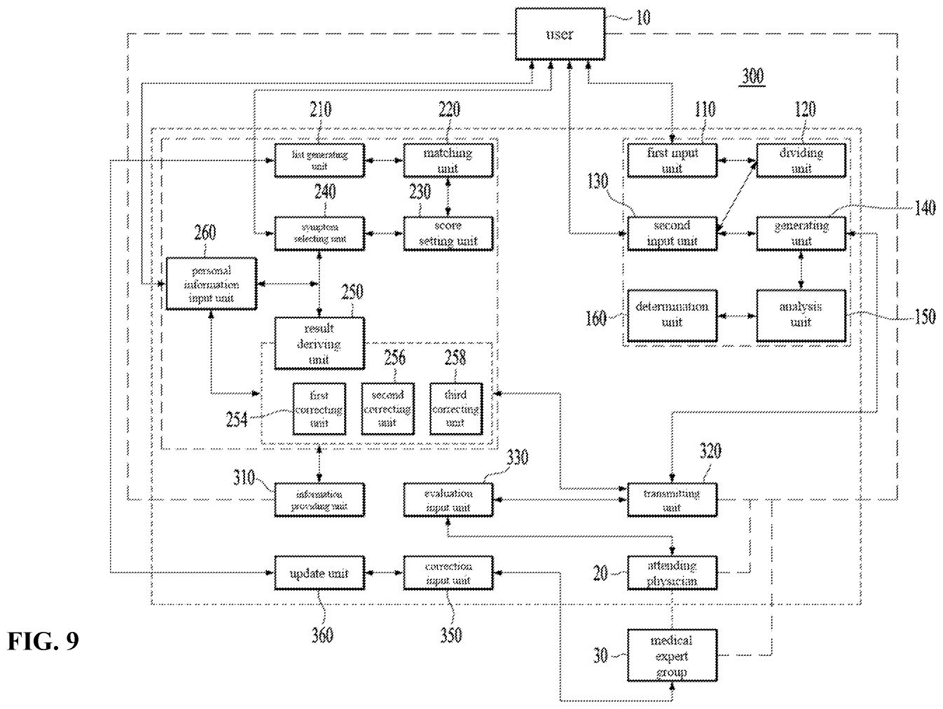

As illustrated in FIG. 9, this embodiment provides a method for providing basis data for diagnosis including a list generating unit 210 that prepares a list of multiple symptoms and a list of multiple diseases, respectively, a matching unit 220 that matches the symptoms with the diseases related to the symptoms, a score setting unit 230 that differentially sets correlation scores for the symptoms matched with the diseases according to prevalence rates of the diseases for the symptoms, for each disease, a symptom selecting unit 240 that provides a user with the list of multiple symptoms and receives, from the user, at least one selection of the symptoms that correspond to the user, a result deriving unit 250 that derives predicted disease information in an order of a highest sum score by summing up scores related to the symptoms selected by the user for each disease and calculating the sum score, an information providing unit 310 that provides the selection result of the user and the predicted disease information to the user, and a transmitting unit 320 that transmits to an electronic medical record (EMR) of an attending physician of the user the list of the multiple symptoms including the selection result of the user and the predicted disease information.

According to this embodiment, each symptom of the list of multiple symptoms is matched with any relevant disease of the list of multiple diseases in the matching unit 220, and in this case, the correlation score of one symptom is differentially set for each disease according to the prevalence rate between the symptom and the disease in the score setting unit 230, and the predicted disease information is derived, in the result deriving unit 250, based on the sum score of the respective correlation scores for each disease according to the result of the symptom selected by the user in the symptom selecting unit 240, and the derived predicted disease information is provided to the user through the information providing unit 310. Accordingly, it is possible for the user to effectively predict and identify his/her disease. Furthermore, by transmitting, in the transmitting unit 320, the list of multiple symptom including the selection result of the user and the predicted disease information to EMR of the attending physician of the user for using as basis data for diagnosis, it is possible to provide faster and more accurate treatment.

Hereinafter, each configuration of the basis data system for diagnosis according to the present embodiment will be described with reference to FIG. 9.

FIG. 9 is a block diagram illustrating a system for providing basic data for diagnosis according to a sixth embodiment of the present invention.

The list generating unit 210 may prepare a list of multiple symptoms and a list of multiple diseases, respectively.

The matching unit 220 may match a symptom with a disease related to the symptom.

The score setting unit 230 may differentially set a correlation score for a symptom matched with a disease for each disease according to a prevalence rate of the disease with respect to the symptom.

The symptom selecting unit 240 may provide the list of multiple symptoms to the user so that at least one symptom corresponding to the user may be selected by the user.

After the step of differentially setting for each disease, the personal information input unit 260 may receive personal information including at least one of gender, age, and residential area from the user.

The result deriving unit 250 may derive the predicted disease information in the order of the highest sum score by summing up the correlation scores of the symptoms selected by the user for each disease and calculating the sum score.

The result deriving unit 250 may include the first correcting unit 254 that corrects the sum score by subtracting a rule out score from the sum score of the disease when the prevalence rate of the disease for the personal information is less than a first criterion.

The result deriving unit 250 may include the second correcting unit 256 that corrects the sum score by subtracting an adjustment score from the sum score of the disease when the prevalence rate of the disease according to the personal information is less than a second criterion.

The result deriving unit 250 may include the third correcting unit 258 that corrects the sum score by subtracting a correction score from the sum score when the deviation between the prevalence rate of the disease due to the personal information and the prevalence rate of the disease due to causes other than the personal information is less than a third criterion.

The symptoms may include a main symptom and a sub symptom for the main symptom.

The result deriving unit 250 may select the top N diseases as the predicted disease information based on the sum score.

A transmitting unit 320 may transmit the list of multiple symptoms including the selection result of the user and the predicted disease information to the EMR of the attending physician of the user.

The system further includes the first input unit 110 that receives information on a discomfort start point from a user, the dividing unit 120 that divides a discomfort period from the discomfort start point to a present point into a plurality of discomfort sections, the second input unit 130 that receives from the user discomfort information obtained by qualifying discomfort levels for the respective discomfort sections, and the generating unit 140 that generates a discomfort graph showing a change in the user's discomfort during the discomfort period based on the discomfort information. The transmitting unit 320 may further transmit the discomfort graph to the EMR of the attending physician.

Each step thereof will be described as below.

The first input unit 110 may receive information on the discomfort start point from the user.

The dividing unit 120 may divide the discomfort period from the discomfort start point to the present point into a plurality of discomfort sections.

Here, the discomfort section may be divided into shorter lengths as it approaches the present point.

More specifically, the plurality of discomfort sections may include a first discomfort section divided from the discomfort start point to 50% or less of the discomfort period as a period representing a reference value for the user's discomfort, a second discomfort section divided from 50% of the discomfort period to 75% or less of the discomfort period as a period representing the average value of the user's discomfort, a third discomfort section divided from 75% of the discomfort period to 90% or less of the discomfort period as a period representing the patten of change in the user's discomfort, and a fourth discomfort section divided from 90% of the discomfort period to the present point as a period representing the current discomfort state of the user.

The second input unit 130 may receive the quantified discomfort information on the discomfort level for each discomfort section from the user.

More specifically, the second input unit 130 may allow the user to select from among discomfort levels classified on a scale of 1 to 10.

An analysis unit 150 may analyze the second to fourth discomfort sections of the discomfort graph to determine whether the user is in an emergency situation.

The analysis unit 150 may analyze the discomfort graph by calculating an average slope of the discomfort graph.

The analysis unit 150 calculates a first slope with respect to the numerical value of the discomfort information of the second discomfort section to the third discomfort section, and calculates a second slope with respect to the numerical value of the discomfort information of the third discomfort section to the fourth discomfort section. Thus, the discomfort graph can be analyzed by calculating the absolute value of the difference between the first slope and the second slope.

A determination unit 160 may determine an emergency situation when the value calculated by the analysis unit 150 analyzing the discomfort graph exceeds a reference value.

The determination unit 160 may determine an emergency situation when the discomfort information of the discomfort section including the present point is greater than or equal to a reference value.

The transmitting unit 320 may transmit the discomfort graph to the EMR of the attending physician of the user.

The transmitting unit 320 may further transmit the list of the multiple symptoms including the selection result of the user and the predicted disease information to the EMR of an attending physician of the user.

An evaluation input unit 330 may receive from the attending physician a result on whether or not the list of multiple symptoms meets an evaluation criteria.

More particularly, whether or not the evaluation criteria is met may be determined based on the diagnosis result for the user by the attending physician.

In addition, after the step of receiving from the attending physician the result, the transmitting unit 320 may transmit the list of multiple symptoms to a server of a designated medical expert group if the list of multiple symptoms does not meet the evaluation criteria. A correction input unit 350 may receive a correction item for the list of multiple symptoms from the medical expert group.

More particularly, the evaluation input unit 330 may receive an opinion of the attending physician regarding the result below the evaluation criteria if the list of multiple symptoms does not meet the evaluation criteria, and the transmitting unit 320 may further transmit the opinion to the server of the medical expert group.

An update unit 360 may update the list of multiple symptoms by correcting the list of multiple symptoms according to the correction item.

Meanwhile, the components of the above-described embodiment may be easily understood from a process point of view. That is, each component can be identified as each process. In addition, the process of the above-described embodiment may be easily understood from the point of view of the components of an apparatus.

In addition, the technical matters described above may be implemented in the form of program instructions that can be executed through various computer means and stored in a computer readable medium. The computer readable medium may include program instructions, data files, data structures, etc. alone or in combination. The program instructions stored on the medium may be specially designed and configured for the embodiments, or may be known and available to those skilled in the art of computer software. Examples of the computer readable storage medium include magnetic media such as hard disk, floppy disk and magnetic tape, optical media such as CD-ROM and DVD, magnetic-optical media such as floptical disk, and hardware devices specially configured to store and execute program instructions such as ROM, RAM, flash memory, and the like. Examples of program instructions include not only machine language codes such as those generated by a compiler, but also high-level language codes that can be executed by a computer using an interpreter or the like. A hardware device may be configured to operate as one or more software modules to perform the operations of the embodiments, and vice versa.

In the above, although an embodiment of the present invention has been described, those of ordinary skill in the art can variously modify and change the present invention by adding, changing, deleting or including components within the scope that does not depart from the spirit of the present invention described in the claims, and this will also be said to be included within the scope of the present invention.

1. A method for generating a discomfort graph comprising the steps of:
receiving information on a discomfort start point from a user;
dividing a discomfort period from the discomfort start point to a present point into a plurality of discomfort sections;
receiving from the user discomfort information obtained by quantifying discomfort levels for the respective discomfort sections; and
generating a discomfort graph showing a change in the user's discomfort during the discomfort period based on the discomfort information.

2. The method according to claim 1, wherein the plurality of discomfort sections includes:
a first discomfort section divided from the discomfort start point to 50% or less of the discomfort period as a period representing a reference value for the user's discomfort;
a second discomfort section divided from 50% of the discomfort period to 75% or less of the discomfort period as a period representing an average value of the user's discomfort;
a third discomfort section divided from 75% of the discomfort period to 90% or less of the discomfort period as a period representing a patten of change in the user's discomfort; and
a fourth discomfort section divided from 90% of the discomfort period to the present point as a period representing a current discomfort state of the user.

3. The method according to claim 2, further comprising, after the step of generating a discomfort graph, the step of analyzing the second to fourth discomfort sections of the discomfort graph to determine whether the user is in an emergency situation.

4. The method according to claim 3, wherein the step of analyzing the discomfort graph is performed by calculating an average slope of the discomfort graph.

5. The method according to claim 3, wherein the step of analyzing the discomfort graph includes the steps of:
calculating a first slope with respect to a numerical value of the discomfort information of the second discomfort section to the third discomfort section;
calculating a second slope with respect to a numerical value of the discomfort information of the third discomfort section to the fourth discomfort section; and
calculating an absolute value of a difference between the first slope and the second slope.

6. The method according to claim 4, further comprising, after the step of analyzing the discomfort graph, the step of determining an emergency situation when the value calculated by analyzing the discomfort graph exceeds a reference value.

7. The method according to claim 1, wherein the step of receiving discomfort information is performed by allowing the user to select one of the discomfort levels classified on a scale of 1 to 10.

8. The method according to claim 1, further comprising, after the step of receiving discomfort information, the step of determining an emergency situation when the discomfort information of the discomfort section including the present point is greater than or equal to a reference value.

9. The method according to claim 1, further comprising, after the step of generating a discomfort graph, the step of transmitting the discomfort graph to an electronic medical record (EMR) of an attending physician of the user.

10. A system for generating a discomfort graph, comprising:
a first input unit that receives information on a discomfort start point from a user;
a dividing unit that divides a discomfort period from the discomfort start point to a present point into a plurality of discomfort sections;
a second input unit that receives from the user discomfort information obtained by qualifying discomfort levels for the respective discomfort sections; and
a generating unit that generates a discomfort graph showing a change in the user's discomfort during the discomfort period based on the discomfort information.

11. The method according to claim 5, further comprising, after the step of analyzing the discomfort graph, the step of determining an emergency situation when the value calculated by analyzing the discomfort graph exceeds a reference value.
